(19) United States
(12) Patent Application Publication
(10) Pub. No.: US 2007/0173716 A1
(43) Pub. Date: Jul. 26, 2007

(54) Method for planning an examination of a subject in an imaging system

(76) Inventors: Klaus Mayer, Eckental (DE); Cecile Mohr, Erlangen (DE); Mike Muller, Mohrendorf (DE)

Correspondence Address: SCHIFF HARDIN, LLP;PATENT DEPARTMENT, 6600 SEARS TOWER, CHICAGO, IL, 60606-6473, US

(21) Appl. No.: 11/482,428

(22) Filed: Jul. 7, 2006

(30) Foreign Application Priority Data
Jul. 7, 2005 (DE) 10 2005 031 902.5

(51) Int. Cl. A61B 5/05

(52) U.S. Cl. 600/410

(57) ABSTRACT

In a method for planning an examination of an examination subject in the magnetic resonance system, wherein images of the examination subject are acquired at different table positions on which the examination subject rests, at least one overview image is generated with which further images in the examination subject are planned, the position of at least one first image in the examination subject is determined that is acquired at a first table position, the measurement parameters for the (at least one) first image at the first table position are established, the position of (at least one) second image in the examination subject is determined, with the (at least one) second image being acquired at a second table position, the measurement parameters for the (at least one) second image at the second table position, , are established and the position of the (at least one) second image in the examination subject and the associated measurement parameters are determined at the second table position for the (at least one) second image before the acquisition of the (at least one) first image.

### BACKGROUND OF THE INVENTION

1. Field of the Invention

The present invention concerns a method for planning an examination of an examination subject in a magnetic resonance system. The invention is particularly (but not exclusively) applicable in magnetic resonance systems that use relatively short magnets for generation of the polarization field B₀, but the invention can be used in magnetic resonance systems with any magnet shape.

2. Description of the Prior Art

In the development of magnetic resonance systems a trend toward ever-shorter magnet designs has emerged. Among other things, this is for the purpose of allowing the examination subject to be examined in a closed magnet system, thus with a magnet that exhibits a higher field strength, which can lead to complications for patients with claustrophobia. The examination of many claustrophobic patients is not possible in closed magnetic resonance systems since these patients are made uncomfortable by the crowded spatial proportions in such magnets. For this reason ever-shorter magnets have been developed so that, for examinations that are not conducted at the head of the patient, the patient looks out from the magnet. These shorter magnets also, however, lead to the situation that the available field of view (FOV) for the acquisition (what is known as the intrinsic field of view (IFOV)) becomes ever smaller. In MR examinations the relevant image section of the examined body region thereby likewise becomes ever smaller. It is simultaneously ever more difficult for the apparatus operator to cover larger examination regions with only one examination.

This presents the problem of how to cover (image) an anatomy region that is larger than the intrinsic field of view with a relatively small size (extent). The region to be covered by the examination, which region is larger than the intrinsic field of view, is called the extended field of view (or virtual large field of view (VLFOV)).

Furthermore, MR techniques have been developed that allow a larger region of the body to be examined, by the table on which the examined person rests being shifted through the magnet. The examination of regions of the body that can be larger than the available field of view in the system, can be achieved by execution of various measurements for different table positions.

According to the prior art, a body region that is larger than the available field of view is examined by the acquisition of data from multiple levels or slabs. The body region is thus deconstructed into individual segments. A measurement protocol composed of, for example, a multiple of imaging sequences is implemented at the associated table position in each segment. For example, an examination of the spinal column can be implemented by three different program steps at various table positions. This implementation, however, is very time-consuming in the planning phase. The operator must individually load the measurement protocols at different table positions and adjust the measurement parameters. He or she must open the individual steps of the individual slabs in series at the interactive display, plan the measurement protocol and close this again. The user does not have the possibility to plan a large anatomical region in a single step, which would significantly shorten the planning of the examination.

### SUMMARY OF THE INVENTION

An object of the present invention is to shorten the planning of an examination of an examination subject for examinations in which measurements are acquired with different table positions.

This object is achieved by a method according to the invention, for planning an examination of an examination subject in a magnetic resonance system wherein images of the examination subject are to be acquired at different table positions wherein, at the beginning, an overview image is generated with further images in the examination subject can be planned. The position of at least one first image in the examination subject is subsequently determined, and the image is acquired at a first table position. The measurement parameters-for this one first image or the number of first images are likewise established at the first table position. In a further step, the position of at least one second image in the examination subject is also determined, and this second image is acquired at a second table position, whereby the measurement parameters are likewise established for this at least one second image at the second table position. According to the invention, the position of the second image in the examination subject and the measurement parameters for the second image at the second table position are now determined before the acquisition of the at least one first image. The workflow steps or the measurement protocols and their measurement parameters for measurements at different table positions are thus determined before the beginning of the measurement. This means that the operator of the magnetic resonance system plans an examination of the examination subject in a single step, with the field of view being larger than the intrinsic field of view. The operator plans an examination in a larger field of view, namely the virtual large field of view. With this method, a larger field of view than is physically, actually present is virtually realized in the planning.

The position of the (at least one) second image preferably is determined at the table position at which the overview image was generated.

Furthermore, the measurement parameters used at the first table position can be adopted for the second image or for the second images at the other table position. In this embodiment, the operator thus does not have to establish the position of the image or, respectively, the images. The measurement parameters can be adopted from the measurement protocol that was executed at the first table position. Naturally it is also possible to alter the measurement parameters at the second table position relative to the measurement parameters at the first position, for example in order to adapt them to the different anatomical geometry. If the operator plans an examination with multiple table feeds, because the acquired overview image does not completely show the second region in the planning, the operating personnel thus can establish the positions of the images in the examined person as follows. The second table position can then be determined such that a specific table feed is established that determines the displacement (shift) of the table relative to the first table position. If the operator were to make exposures of a very large anatomical region, still further images can naturally be acquired at further table positions, and for this purpose only the table feed relative to the respective last measurement must be determined. The operator can consequently plan all slabs, in the extreme case the entire body, at once, and can adjust all protocol parameters of all measurement protocols involved in the measurement individually in one step.

Furthermore, it is possible for a composite image composed of the first image and the second image to be automatically generated after implementation of the measurement at the first table position and at the second table position. This model (known as a composite model that can be generated as described above), or any other composite model that is composed from images at different table positions, can likewise be used as a model in order to plan an examination. The operating personnel of the magnetic resonance system thus can directly plan the images on the composite image by determining the position of the first image and the position of the second image in the composite image. The specification of the table feed is not absolutely necessary. The MR system can calculate the second table position from the position of the second image relative to the first image. It is in turn possible to automatically assemble the images that result from the measurement (which was generated with the aid of the composite model) after the acquisition, such that a further composite image arises.

### DESCRIPTION OF THE DRAWINGS

FIG. 1 is a flowchart for planning of a measurement at different table positions in accordance with the invention.

FIG. 2 shows schematically the position of the regions to be examined with the associated fields of view on a patient.

FIG. 3 is a flowchart for planning on a composite model in accordance with the invention.

### DESCRIPTION OF THE PREFERRED EMBODIMENTS

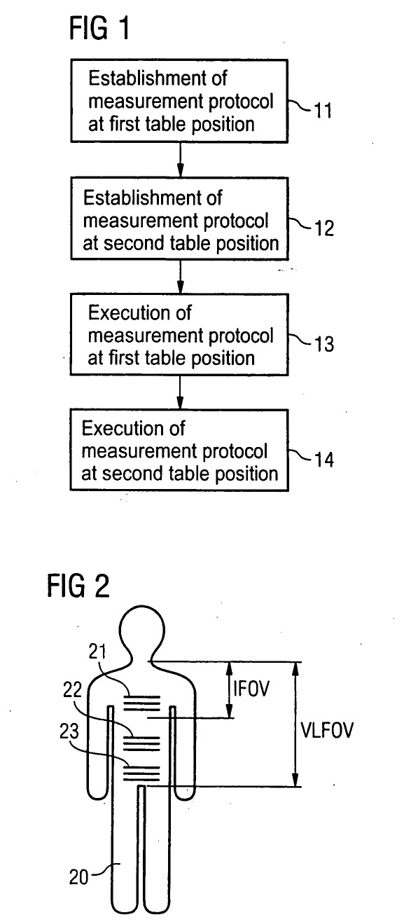

Basic steps for implementation of a planning of a measurement with different table positions are shown in FIG. 1. As shown in FIG. 2, for example, an acquisition of a region of the body (for example the spinal column) is to be made, whereby the total region to be examined corresponds to the shown virtual large field of view (VLFOV). The available viewing field, however, is only the shown field of view or intrinsic field of view (IFOV). An overview exposure or overview image of this intrinsic field of view now exists that can be used in order to plan the further measurement. For this purpose, the positions at which images should be acquired in the shown examination person 20 can be determined. For example, three sets of slices can be planned in an existing model image in FIG. 2.

Referring to FIG. 1, this means that the measurement protocol is established at a first table position in a step 11. The positions of the slices 21 in the examination person must hereby be established as well as the image acquisition parameters such as matrix size, echo time, repetition time etc. Furthermore, in a step 12 the measurement protocol for the second table position is established in that the position of the slices 22 in the examination person is established. This can occur, for example, by determining a table feed in centimeters with which the measurements are repeated that were generated with the first measurement protocol. In a step 13, the measurement protocol then can be executed at the first table position before the measurement protocol is executed at the second table position in a step 14 to measure the slices 22. An intervention of the operating personnel between the steps 13 and 14 is no longer necessary; the operating personnel can plan and set all slabs (meaning the measurement protocols at the different table positions) at once. The loading of the individual measurements in series at the various table positions, as is necessary in the prior art and the individual planning of the measurements at the different table positions, is not needed. The examination personnel then can implement a further (last) measurement at the third table position in order to acquire the data for the slices 23.

The user can plan an examination in one step as shown in FIG. 2 and 1 with a single protocol, what is known as a multi-step protocol. This can in particular significantly accelerate the planning of examinations such as, for example, those of the entire spinal column or of the entire body. The method can likewise be used in magnetic resonance angiographies, normally in whole-body angiographies or in peripheral angiographies. The search for metastases in a screening examination is likewise made easier since for the most part a large body region must be examined to search for these metastases. This is made easier by a single planning step that includes a number of measurement protocols at different table positions.

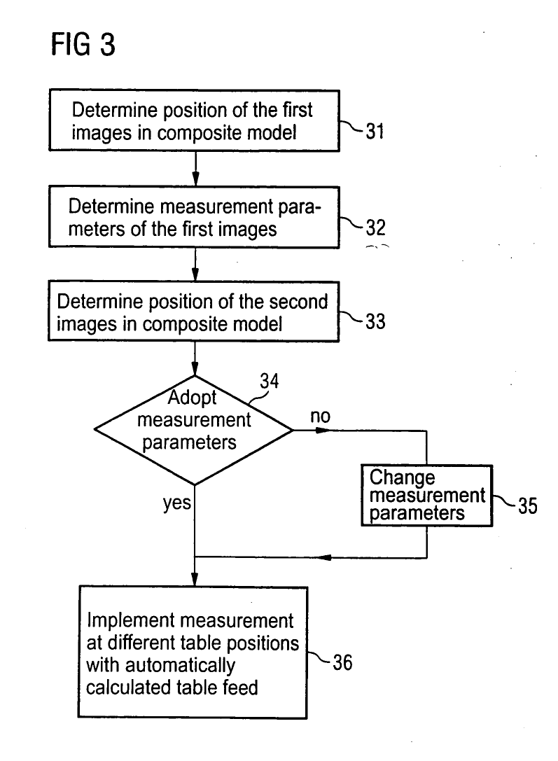

Furthermore, the method steps for planning an examination when a composite image exists are shown in FIG. 3. This composite image can be calculated when images are acquired at different table positions and are subsequently assembled via post-processing such that an MR image results that shows a region of the body that is larger than the field of view available in a measurement. In a first step, the position of the first image sequence can be determined in the composite model. In the present invention, for the most part multiple first images are spoken of since for the most part a plurality of adjacent slices of an anatomical region is acquired. It is naturally also possible for only a single slice or only one image to be acquired (step 31). The measurement parameters for the first image or the first images is/are likewise determined in a step 32. Since a composite model is now present on which a larger region of the examined body is shown, in a step 33 the second images can be positioned, for example, so that the position of the second image or the second images is/are determined in the composite model. In a step 34 it is subsequently checked whether the measurement parameters determined in step 32 should also be adopted for the second images. If this is not the case, the measurement parameters can be adapted as necessary (step 35). If the measurement parameters can be adopted unchanged, the examination is subsequently conducted entirely in a step 36, meaning that the first images are acquired at the first table position and the second images are acquired at the second position. The system can also automatically calculate the table feed from the relative position of the first images with regard to the second images, such that the input of a table feed by the operating personnel is not necessary. This makes the planning of a measurement on a composite image easier in that the different positions of the images can be determined in a single step.

In summary, the present invention enables the examination of a larger anatomical region with a VLFOV with only a single contiguous planning step. The individual invocations of the measurement protocols at the different table positions and the planning of the different measurements is no longer necessary. This leads to a reduced operator effort in the measurement preparation. The inconvenient processing of a number of individual measurement protocols for large examination regions is avoided. The planning is in particular possible and made easier on a composite model image.

Although modifications and changes may be suggested by those skilled in the art, it is the intention of the inventors to embody within the patent warranted hereon all changes and modifications as reasonably and properly come within the scope of their contribution to the art.

We claim as our invention:

1. A method for planning an examination of an examination subject in a magnetic resonance imaging system having a magnetic resonance data acquisition device with a patient table, adapted to receive the examination subject thereon, movable through a plurality of different table positions in the data acquisition device, said method comprising the steps of:
with said data acquisition system, generating an overview image of the examination subject, from which subsequent diagnostic images of the examination subject are planned;
in the overview image, determining a position of a first of said diagnostic images in the examination subject that will be acquired with the patient table at a first table position;
establishing data acquisition parameters for operating said data acquisition system to acquire said first of said diagnostic images at said first table position;
before acquiring said first of said diagnostic images, determining a position in the overview image of a second of said diagnostic images in the examination subject, said second of said diagnostic images to be acquired with said patient table at a second table position; and
establishing data acquisition parameters for acquiring said second of said diagnostic images at said second table position.

2. A method as claimed in claim 1 comprising selecting said second table position for acquiring said second of said diagnostic images as the same table position at which said overview image was acquired.

3. A method as claimed in claim 1 comprising adopting said data acquisition parameters for acquiring said first of said diagnostic images at said first table position to acquire said second of said diagnostic images at said second table position.

4. A method as claimed in claim 1 comprising moving said patient table through said data acquisition system with a table feed, and determining said second table position relative to said first table position dependent on said table feed.

5. A method as claimed in claim 1 comprising automatically generating a composite image from said first of said diagnostic images and said second of said diagnostic images.

6. A method as claimed in claim 1 comprising generating a composite model from images of the examination subject at different table positions of said patient table, and determining a position of said first of said diagnostic images and said second of said diagnostic images in said composite image by manual interaction with said composite image, and automatically electronically calculating said second table position from the position of said second of said diagnostic images in said composite image.

7. A method as claimed in claim 6 comprising generating said composite model at least from said first of said diagnostic images and said second of said diagnostic images.
